(19) United States
(12) Patent Application Publication
(10) Pub. No.: US 2014/0276361 A1
(43) Pub. Date: Sep. 18, 2014

(54) SYSTEMS AND METHODS FOR TREATING GLAUCOMA

(71) Applicant: AVEDRO, INC., WALTHAM, MA (US)

(72) Inventors: Satish Herekar, Palo Alto, CA (US); Marc D. Friedman, Needham, MA (US); David Muller, Boston, MA (US)

(73) Assignee: AVEDRO, INC., WALTHAM, MA (US)

(21) Appl. No.: 14/216,392

(22) Filed: Mar. 17, 2014

Related U.S. Application Data
(63) Continuation-in-part of application No. PCT/US2013/071080, filed on Nov. 20, 2013.
(60) Provisional application No. 61/728,789, filed on Nov. 20, 2012.
(60) Provisional application No. 61/792,463, filed on Mar. 15, 2013.

(51) Int. Cl. A61N 5/06; A61B 19/00; A61F 9/00; A61K 31/525; A61K 31/352

(52) U.S. Cl. CPC A61N 5/062; A61K 31/525; A61K 31/352; A61F 9/0017; A61B 19/5225
USPC 604/21

(57) ABSTRACT

A glaucoma treatment system includes: a cannula body configured to be positioned in an area of Schlemm's canal; an illumination guide extending along the cannula body; at least one drug source coupled to the cannula body; a cross-linking agent source coupled to the cannula body; and an illumination source coupled to the illumination guide. The at least one drug source includes a drug that promotes outflow of aqueous humor through the trabecular meshwork and into Schlemm's canal. The cannula body delivers the drug from the at least one drug source to the area of Schlemm's canal, and in response to changes in the outflow of aqueous humor, delivers the cross-linking agent to the area of Schlemm's canal. The illumination guide delivers photo-activating light from the illumination source to the area of Schlemm's canal. The photo-activating light activates the cross-linking agent, thereby stabilizing changes in the area of Schlemm's canal.

### CROSS REFERENCE TO RELATED APPLICATIONS

This application is a continuation-in-part application of PCT Application No. PCT/US2013/071080, filed on Nov. 20, 2013, which claims priority to U.S. Provisional Application No. 61/728,789, filed Nov. 20, 2012. This application also claims priority to U.S. Provisional Patent Application No. 61/792,463, filed Mar. 15, 2013. The contents of these applications are incorporated entirely herein by reference.

### FIELD OF THE INVENTION

The present invention relates generally to systems and methods for treating glaucoma, and more particularly, to systems and methods for generating cross-linking activity in areas of the eye, such as the lamina cribrosa and/or peripapillary sclera, to treat glaucoma.

### BACKGROUND

Glaucoma refers to a group of eye conditions that lead to damage to the optic nerve. This nerve carries visual information from the eye to the brain. In most cases, damage to the optic nerve results from increased pressure in the eye, also known as intraocular pressure (IOP). Liquid aqueous humor is continuously produced by the ciliary processes of the eye. The aqueous humor is filtered through the trabecular meshwork and then drained through Schlemm's canal into scleral plexuses and general blood circulation. When too much aqueous humor is made, or when it is not drained sufficiently, the intraocular pressure rises. This build-up of aqueous humor can lead to glaucoma. Generally, treatment of glaucoma involves efforts to reduce intraocular pressure.

Prior studies have determined that the most significant risk factors for the development of glaucoma include age, IOP, cup/disc ratio, and thin central corneal thickness. The identification of central corneal thickness as a risk factor has generated increased interest in the biomechanical properties of the ocular coat and its role in the pathophysiology of glaucoma.

The nerve fibers forming the optic nerve exit the eye posteriorly through a hole in the sclera that is occupied by a mesh-like structure called the lamina cribrosa. It is formed by a multilayered network of collagen fibers that insert into the scleral canal wall. The nerve fibers that comprise the optic nerve run through pores formed by these collagen beams. The lamina cribrosa helps maintain the pressure gradient between the inside of the eye and the surrounding tissue. Due to IOP, the lamina cribrosa bulges slightly outwards. Being structurally weaker than the much thicker and denser sclera, the lamina cribrosa is more sensitive to changes in the intraocular pressure and tends to react to increased pressure through posterior displacement. This is thought to be one of the causes of nerve damage in glaucoma, as the displacement of the lamina cribrosa causes the pores to deform and pinch the traversing nerve fibers and blood vessels.

According to U.S. Pat. App. Pub. No. 2010/0189817 to Krueger et al. (the contents of which are incorporated entirely herein by reference), collagen cross-linking of the lamina cribrosa and/or peripapillary sclera provides a method for modulating biomechanical stress and strain-based injury mechanisms in the laminar region toward the goal of preventing the onset of, or slowing the progression of, glaucomatous optic neuropathy. IOP elevation measurably increases the in situ stiffness of the optic nerve/lamina cribrosa and peripapillary sclera and is accompanied by circumferential strain in both regions. Krueger et al. observes that collagen cross-linking of the peripapillary sclera measurably stiffens the peripapillary sclera and buffers the optic nerve/lamina cribrosa from stiffening and circumferential strain during IOP elevation. These observations suggest approaches for modifying stress and strain-based mechanisms of injury in glaucomatous optic neuropathy.

### SUMMARY

Embodiments according to aspects of the present invention provide systems and methods for generating cross-linking activity in areas of the eye, such as the lamina cribrosa and/or peripapillary sclera, to treat glaucoma.

In one example embodiment, a system for treating glaucoma includes a catheter configured for insertion into an eye, the catheter having a proximal end and a distal end and including: a lumen configured to be coupled, at the proximal end, to a cross-linking agent source containing a cross-linking agent formulation. The system also includes an optical fiber configured to be coupled, at the proximal end, to a photoactivating light source. In addition, the system includes a guide that is operable from the proximal end to direct the distal end of the catheter to selected eye tissue in a lamina cribrosa, a peripapillary sclera, or a combination of the lamina cribrosa and the peripapillary sclera of the eye as the catheter is inserted into the eye. The lumen delivers the cross-linking agent formulation from the cross-linking agent source directly to the selected eye tissue at the distal end of the catheter, and the optical fiber delivers photoactivating light from the photoactivating light source directly to the selected eye tissue treated with the cross-linking agent to generate cross-linking activity in the selected eye tissue.

In another example embodiment, a system for treating glaucoma includes a cross-linking agent system including a cross-linking agent source containing a cross-linking agent formulation and a cross-linking agent delivery device, the cross-linking agent delivery device being coupled to the cross-linking agent source, the cross-linking agent delivery device being configured for insertion into the eye and to deliver the cross-linking agent formulation from the cross-linking agent source directly to selected eye tissue in a lamina cribrosa, a peripapillary sclera, or a combination of the lamina cribrosa and the peripapillary sclera of an eye. The system also includes an activation system including a photoactivating light source and a photoactivating light delivery device configured to deliver photoactivating light to the selected eye tissue treated with the cross-linking agent formulation to generate cross-linking activity in the selected eye tissue. In some cases, the activation system is configured to deliver the photoactivating light from outside the eye and to a depth of the selected eye tissue below a surface of the eye.

In yet another example embodiment, a system for treating glaucoma includes: a cannula body configured to be positioned in an area of Schlemm's canal; an illumination guide extending along the cannula body; at least one drug source coupled to the cannula body; a cross-linking agent source coupled to the cannula body; and an illumination source coupled to the illumination guide. The at least one drug source includes a drug that promotes outflow of aqueous humor through the trabecular meshwork and into Schlemm's canal. The cannula body delivers the drug from the at least one drug source to the area of Schlemm's canal, and in response to changes in the outflow of aqueous humor, delivers the cross-linking agent to the area of Schlemm's canal. The illumination guide delivers photo-activating light from the illumination source to the area of Schlemm's canal after the cross-linking agent has been delivered. The photo-activating light generates cross-linking activity in the area of Schlemm's canal by activating the cross-linking agent, thereby stabilizing changes in the area of Schlemm's canal.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 illustrates an example treatment system that delivers and activates a cross-linking agent to treat glaucoma, according to aspects of the present invention.

FIG. 2 illustrates an example catheter system including a cross-linking agent system and an activation system that facilitate the delivery of a cross-linking agent and photoactivating light to areas of the eye for treatment of glaucoma, according to aspects of the present invention.

FIG. 3 illustrates an example activation system that includes a laser light source, a mirror array, and objective lens to employ laser scanning to initiate cross-linking activity in target eye tissue, according to aspects of the present invention.

FIG. 4 illustrates another example activation system that includes a laser system that employs multiphoton excitation to initiate cross-linking activity in target eye tissue, according to aspects of the present invention.

FIG. 5 illustrates yet another example activation system that includes a laser system that employs single photon excitation to initiate cross-linking activity in target eye tissue, according to aspects of the present invention.

FIG. 6 illustrates a further example of a system that treats glaucoma by delivering at least one drug for promoting aqueous humor outflow through the trabecular meshwork and into Schlemm's canal and generating cross-linking activity to preserve the changes in the area of Schlemm's canal, according to aspects of the present invention.

While the invention is susceptible to various modifications and alternative forms, a specific embodiment thereof has been shown by way of example in the drawings and will herein be described in detail. It should be understood, however, that it is not intended to limit the invention to the particular forms disclosed, but on the contrary, the intention is to cover all modifications, equivalents, and alternatives falling within the spirit of the invention.

### DESCRIPTION

Embodiments according to aspects of the present invention provide systems and methods for generating cross-linking activity in areas of the eye, such as the lamina cribrosa and/or peripapillary sclera, to treat glaucoma.

FIG. 1 illustrates an example treatment system 100 that delivers and activates a cross-linking agent to treat glaucoma according to aspects of the present invention. In particular, the treatment system 100 includes a cross-linking agent system 110 and an activation system 120. The cross-linking agent system 110 includes a source 110a for a cross-linking agent formulation and a cross-linking agent delivery device 110b. The delivery device 110b, which is coupled to the source 110a, delivers the cross-linking agent formulation in step 10 to eye tissue where cross-linking of collagen fibers will strengthen the eye tissue and reduce the effect of IOP on the optic nerve, i.e., the lamina cribrosa and/or peripapillary sclera. In step 20, the activation system 120 delivers an activating element to initiate cross-linking activity in the eye tissue treated with the cross-linking agent formulation in step 10.

The treatment system 100 may also include a monitoring system 130 that may be employed to monitor the operation of the cross-linking agent system 110 and the activation system 120. Additionally, the treatment system 100 may include a controller 140 to control aspects of the operation of the cross-linking agent system 110 and the activation system 120. The controller may be communicatively coupled to the monitoring system 130 to process the images, data, etc., from the monitoring system 130 and to determine any necessary response to such feedback.

As FIG. 1 shows, the cross-linking agent may be a formulation containing riboflavin and the activating element may be ultraviolet (UV) light. In this case, the activation system 120 includes an activating element source 120a that provides UV light and an activating element delivery device 120b that directs the UV light to the treated eye tissue. The element delivery device 120b may employ any optical element(s) to guide the UV light. The UV light initiates cross-linking activity by causing the riboflavin to release reactive riboflavin and oxygen radicals in the eye tissue. The riboflavin acts as a sensitizer to convert O₂ into singlet oxygen along with radical riboflavin, causing cross-linking within the eye tissue.

Cross-linking of collagen fibers in the lamina cribrosa and/or peripapillary sclera modulates biomechanical stress and strain-based injury mechanisms in the laminar region toward the goal of preventing the onset or slowing the progression of glaucomatous optic neuropathy. Collagen cross-linking of the peripapillary sclera measurably stiffens the peripapillary sclera and buffers the optic nerve/lamina cribrosa from stiffening and circumferential strain during IOP elevation. As such, the embodiment of FIG. 1 may be employed to initiate cross-linking in selected portions of eye tissue in the lamina cribrosa and/or peripapillary sclera. Correspondingly, according to aspects of the present invention, the treatment system 100 facilitates the delivery of the cross-linking agent and the activating element to these areas of the eye. It is noted that because the area around the lamina cribrosa and/or peripapillary sclera does not include photoreceptors, there is less concern about the safety of applying photoactivating light, e.g., UV light.

FIG. 2 illustrates a catheter system 200 including an example cross-linking agent system 210 and an example activation system 220. The catheter system 200 includes a proximal end 200a and a distal end 200b. The distal end 200b is inserted into (or about) the eye and guided to target eye tissue 2 of the lamina cribrosa and/or peripapillary sclera. The catheter system 200 may include a guide, which may have one or more guidewires 205 that are operated at the proximal end 200a to steer the distal end 200b to the target eye tissue 2. For example, the one or more guidewires 205 are coupled to the distal end 200b and may be operated to move along the longitudinal axis of the catheter system 200. The axial movement pulls (or pushes) the distal end 200b and causes the distal end 200b to bend away from (or toward) the longitudinal axis in varying degrees. The flexible portions of the catheter system 200 guided through the eye may be formed, for example, from a flexible biocompatible material, such as a nickel titanium alloy (nitinol).

The catheter system 200 may also include a monitoring system 230 that allows an operator to determine the position of the distal end 200b as it is being guided to the target eye tissue 2. Additionally, the catheter system 200 may include a controller 250 (similar to the controller 150) to control aspects of the operation of the catheter system 200. The controller may be communicatively coupled to the monitoring system 230 to process the images, data, etc., from the monitoring system 230 and to determine any necessary response to such feedback.

The cross-linking agent system 210 includes a lumen 210b that is coupled to a cross-linking agent source 210a at the proximal end 200a. A desired dose of cross-linking agent formulation is delivered as a solution from the cross-linking agent source 210a and through the lumen 210b to the target eye tissue 2. For example, the cross-linking agent source 210a may include a syringe with a reservoir that holds the cross-linking agent. The lumen 210b is coupled to the syringe, e.g., via a receiving port. The plunger of the syringe can then be operated to push the dose of cross-linking agent from the reservoir and through the lumen 210b to the target eye tissue 2. In general, however, it is contemplated that any type of manual or automated mechanism, not limited to a syringe, may be employed to move the dose of cross-linking agent from a source 210a and through the lumen 210b to the distal end 200b.

The activation system 220 delivers the activating element to the target eye tissue 2 at the distal end 200b. In this case, the activating element is light, e.g., UV light, that photoactivates the cross-linking agent, e.g., riboflavin. The activation system 220 includes an optical fiber 220b that extends from the proximal end 200a to the distal end 200b. The optical fiber 220b is coupled to a controlled light source 220a at the proximal end 200a. The optical fiber 220b delivers a desired dose of light from the light source 220a to the distal end 200b to initiate the desired cross-linking activity at the target eye tissue 2. The activation system 220 may provide light according to any combination of: wavelength, bandwidth, intensity, power, duration of treatment, etc., to initiate desired cross-linking activity. In some embodiments, the optical fiber 220b is disposed in, and extends through, the lumen 210b as shown in FIG. 2(A). In alternative embodiments, the optical fiber 220b may be disposed outside the lumen 210b, e.g., in a second lumen 211 (e.g., catheter sheath) that contains both the lumen 210b and the optical fiber 220b as shown in FIG. 2(B).

Because the lumen 210b and the optical fiber 220b both extend to the same distal end 200b of the catheter system 200, the photoactivating light is advantageously delivered directly to an area that generally coincides with the area where the cross-linking agent formulation has been delivered by the lumen 210b. As such, the catheter device 200 does not have to be repositioned between the delivery of the cross-linking agent formulation and the delivery of the photoactivating light. In other words, the photoactivating light is delivered precisely to the treated eye tissue without requiring the additional step of positioning a device to deliver the photoactivating light.

As described above, the catheter system 200 may include a monitoring system 230. In one embodiment, the monitoring system 230 includes an imaging system 232 that captures images, e.g., video, of the area around the distal end 200b. The images from the distal end 200b may be captured by a camera and the images can be transmitted to a display. Image signals from the distal end 200b can be transmitted by a cable that extends from the proximal end 200a to the distal end 200b. Additionally, any illumination required to capture the images may also be delivered to the distal end 200b, e.g., by optical fiber. An operator monitors the images to ensure that the distal end 200b is positioned properly at the target eye tissue 2 and/or that the cross-linking agent and the photoactivating light are properly delivered by the catheter system 200.

The monitoring system 230 may employ additional or alternative approaches for monitoring the operation of the catheter system 200 and/or the treatment applied to the target eye tissue 2. For example, in some embodiments, the monitoring system 230 includes an optical coherence tomography (OCT) system 234 to generate a three-dimensional image of the target eye tissue 2. The OCT system 234 generally utilizes low coherence interferometry of white optical light or near-infrared light. In contrast to coherent interferometry techniques with long coherence lengths (e.g., those utilizing laser light sources), interference in the OCT system 234 is shortened to a distance of micrometers, due to the use of broadband light sources (e.g., sources that can emit light over a broad range of frequencies). Light in the OCT system is broken into two beams: a sample beam, which is directed toward the eye, and a reference beam, which is directed toward a reference surface. The combination of reflected light from the eye and the reference surface are interfered to produce an interference pattern. Constructive interference generally occurs only if light from the two beams travel an optical distance within a coherence length. By scanning the reference surface (e.g., a reference mirror) a reflectivity profile of the eye can be obtained at different depths of the eye tissue 2. Generally, areas of the eye that reflect back a significant amount of light will create greater interference than areas that do not. Any light that is outside the short coherence length will not interfere. Thus, adjusting the reference surface allows the OCT system 234 to be tuned to particular depths of the eye. Such a reflectivity profile (“interference pattern”) is referred to as an A-scan. These axial depth scans (A-scans) can be laterally combined to create a cross-sectional tomography (B-scan). The OCT system 234 thus provides a high resolution (micrometer scale) three-dimensional (to millimeter depths) profile of the eye tissue 2. In particular, the OCT system 234 can be tuned to provide a profile of the areas treated by the catheter system 200.

While the OCT system 234 is described above as a time domain OCT, which scans depths of the eye during distinct time intervals, this is for illustrative purposes only. It is specifically noted that the OCT system 234 can be implemented as one of a variety of available OCT systems, including frequency domain OCT, spectral domain OCT, Fourier domain OCT, time encoded frequency domain OCT, and swept source OCT. Generally, a frequency domain OCT system operates by performing Fourier transforms on the received data to identify the contributions from the returning signal corresponding to different depths in the eye tissue 2. A frequency domain OCT generally is able to generate a full three-dimensional model of the eye in less time compared to a time domain OCT, because the position of the reference arm is not adjusted. Frequency domain OCT systems can be implemented with spatially encoded detectors utilizing, for example, gratings situated in front of CCD detector arrays to distinctly detect different wavelengths of the returning signal via different regions of the CCD detector array. Time encoded frequency domain OCT are implemented with a reference light source that has a characteristic frequency which changes in time. Thus, in a time encoded frequency domain OCT, the eye is probed according to varying wavelengths of light, and the returning signals therefore correspond to varying depths of the eye tissue 2.

The various implementations of the OCT system 234 offer different performance criteria in the form of scan depth, axial resolution, speed of measurement, and signal to noise ratio. These performance criteria may influence a designer's choice of system. For example, implementing the OCT system 234 as a frequency domain OCT system may be desirable because a frequency domain OCT system offers enhanced measurement speed and can generate a full three-dimensional model of the eye without modifying physical features of the OCT system 234 (such as the position of the reference surface). In general, the various OCT systems each are operable to generate three-dimensional profiles of the eye, which can be employed to monitor the positioning and/or operation of the catheter system 200. An example of an OCT system is the Stratus OCT™ (Carl Zeiss Meditec, Inc.).

Dynamically gathering three-dimensional profiles of the treated eye tissue 2 using the OCT system 234 also allows the effect of the cross-linking activity to be precisely characterized at a high resolution. For example, using the OCT system 234, the changes to biomechanical strength of the eye tissue 2 due to the cross-linking treatment can be observed. For example, the pre-treatment response and post-treatment response of the eye tissue 2 to changes in intraocular pressure over the course of a cardiac pulse cycle can provide a useful indicator of changes to the biomechanical strength. In some embodiments, a force can be applied to the eye tissue 2, e.g., via ultrasound pressure waves, and the OCT system 234 is used to determine the effect of the force on the eye to observe the biomechanical strength of the eye tissue 2. The effect of the force on the eye can be determined by measuring an amount of deformation caused by the force or a rate of recovery from the deformation in response to the force.

It is understood that the monitoring system 230 may employ other approaches for monitoring the operation of the catheter system 200 and/or the treatment applied to the target eye tissue 2. For example, in other embodiments, an ultrasound imaging system 236 may be employed to generate images of the target eye tissue 2.

In the embodiment of FIG. 2, the lumen 210b of the catheter system 200 delivers a desired dose of cross-linking agent, e.g., riboflavin, directly to aspects of the lamina cribrosa and/or peripapillary sclera, and the optical fiber 220b delivers photoactivating light directly into the eye to initiate cross-linking in treated areas of eye tissue. In other embodiments, a catheter system, or alternatively a syringe/needle, may deliver the cross-linking agent directly to aspects of the lamina cribrosa and/or peripapillary sclera, but the photoactivating light is delivered through the eye from an external activation system. Aspects of the external activation systems described below may be employed to deliver the photoactivating light from outside the eye and to a depth of the selected eye tissue below a surface of the eye. As with the activation system 220, the activation system provides light according to any combination of: wavelength, bandwidth, intensity, power, duration of treatment, etc., to initiate desired cross-linking activity.

For example, FIG. 3 illustrates an external activation system 320 that employs a configuration of optical elements to direct photo activating light through the eye to the target eye tissue 2, which has been treated with cross-linking agent, e.g., riboflavin. The activation system 320 includes a laser light source 320a and employs laser scanning techniques to deliver the photoactivating light to the target eye tissue 2. For example, the laser light source 320a can be a UV light source that emits a UV laser. A beam of light emitted from the light source 320a passes to a mirror array 320b. Within the mirror array 320b, the beam of light is scanned over multiple mirrors configured in an array. The beam of light can be scanned over the mirrors in the mirror array 320b using, for example, one or more adjustable mirrors to direct the beam of light to point at each mirror in turn. The beam of light can be scanned over each mirror one at a time. Alternatively, the beam of light can be split into one or more additional beams of light using, for example, a beam splitter, and the resultant multiple beams of light can then be simultaneously scanned over multiple mirrors in the mirror array 320b. By rapidly scanning the beam of light over the mirrors in the mirror array 320b, the mirror array 320b outputs a light pattern, which has a two dimensional intensity pattern. The two dimensional intensity pattern of the light pattern is generated by the mirror array 320b according to, for example, the length of time that the beam of light is scanned over each mirror in the mirror array 320b. In particular, the light pattern can be considered a pixelated intensity pattern with each pixel represented by a mirror in the mirror array 320b and the intensity of the light in each pixel of the light pattern proportionate to the length of time the beam of light scans over the mirror in the mirror array 320b corresponding to each pixel. In an implementation where the beam of light scans over each mirror in the mirror array 320b in turn to create the light pattern, the light pattern is properly considered a time-averaged light pattern, as the output of the light pattern at any one particular instant in time may constitute light from as few as a single pixel in the pixelated light pattern. The mirror array 320b may include an array of small oscillating mirrors, controlled by mirror position motors. The mirror position motors can be servo motors for causing the mirrors in the mirror array 320b to rotate so as to alternately reflect the beam of light from the light source 320a toward the eye tissue 2. A controller can control the light pattern generated in the mirror array 320a using the mirror position motors.

The activation system 320 also includes an objective lens 320c which directs the pattern into a small focal volume corresponding to the eye tissue 2. The objective lens 320c determines the depth to which the light pattern form the mirror array 320b is focused. For example, the controller can utilize a position motor to raise and/or lower the objective lens 320c in order to adjust the focal plane of the light pattern. By adjusting the focal plane of the light pattern using the objective lens 320c and controlling the two-dimensional intensity profile of the light pattern using the mirror array 320b, the delivery of the photoactivating light to the eye tissue 2 is controlled in three dimensions. Cross-linking activity is generated three-dimensionally by delivering the UV light to selected regions on successive planes in the treated eye tissue 2. In alternative embodiments, the objective lens 320c can be replaced by an optical train consisting of mirrors and/or lenses to properly focus the light pattern emitted from the mirror array 320b.

Some embodiments may employ Digital Micromirror Device (DMD) technology to modulate the application of initiating light, e.g., UV light, spatially as well as a temporally. Using DMD technology, a controlled light source projects the initiating light in a precise spatial pattern that is created by microscopically small mirrors laid out in a matrix on a semiconductor chip, known as a DMD. Each mirror represents one or more pixels in the pattern of projected light.

FIG. 4 illustrates an external activation system 420 that includes a laser system 422 that directs photoactivating light through the eye to the target eye tissue 2, which has been treated with cross-linking agent, e.g., riboflavin. The activation system 420 may include a docking system 421 that couples the laser system 422 to the eye, e.g., the cornea/sclera. The docking system 421, for example, may employ a vacuum supplied by a vacuum source (not shown) to maintain a stable position on the eye. In turn, the docking system 421 stabilizes the position of the laser system 422 relative to the eye and the target eye tissue 2. The activation device 420 may also include a guidance system 424 to guide the application of the light from the laser system 422. The guidance system 424 may include an OCT system to determine where the activating light from the laser system 422 should be directed.

In other embodiments, other imaging techniques may be employed. For example, the confocal microscopy may be employed to guide the laser system 422. Confocal microscopy is an optical imaging technique used to increase optical resolution and contrast of a micrograph by using point illumination and a spatial pinhole to eliminate out-of-focus light in specimens that are thicker than the focal plane. It enables the reconstruction of three-dimensional structures from the obtained images.

The laser system 422 may employ multiphoton excitation to activate the cross-linking agent. In particular, rather than delivering a single photon of a particular wavelength to the target eye tissue 2, the activation system 420 delivers multiple photons of longer wavelengths and lower energy, which combine to initiate the cross-linking Advantageously, longer wavelengths are scattered within the target eye tissue 2 to a lesser degree than shorter wavelengths, which allows longer wavelengths of light to penetrate the target eye tissue 2 more efficiently than shorter wavelength light. For example, in some embodiments, two photons may be employed, where each photon carries approximately half the energy necessary to excite the molecules in the cross-linking agent that release reactive riboflavin and oxygen radicals. When a cross-linking agent molecule simultaneously absorbs both photons, it absorbs enough energy to release reactive riboflavin and oxygen radicals in the corneal tissue. Embodiments may also utilize lower energy photons such that a cross-linking agent molecule must simultaneously absorb, for example, three, four, or five, photons to release reactive riboflavin and oxygen radicals. The probability of the near-simultaneous absorption of multiple photons is low, so a high flux of excitation photons may be required, and the high flux may be delivered through a femtosecond laser. Because multiple photons are absorbed for activation of the cross-linking agent molecule, the probability for activation increases with intensity. Therefore, more activation occurs where the delivery of light from the laser system 422 is tightly focused compared to where it is more diffuse. Effectively, activation of the cross-linking agent is restricted to the smaller focal volume where the light is delivered to the target eye tissue 2 with a high flux. This localization advantageously allows for more precise and safer control over where cross-linking is activated within the target eye tissue 2. Unlike other multiphoton laser systems that may employ very fast lens systems while applying a laser to other parts of the eye, e.g., the cornea, the laser system 422 may require a slower lens system that allows proper focusing on the target eye tissue 2, particularly, the lamina cribrosa and/or peripapillary sclera.

Although the laser system 422 of the activation system 420 may employ multiphoton excitation, FIG. 5 illustrates an external activation system 520 with a laser system 522 that alternatively employs single photon activation to deliver photoactivating light through the eye and initiate cross-linking activity in the target eye tissue 2. Single photon excitation involves applying photons of a particular wavelength to the target eye tissue 2. In general, the single photon excitation applies higher energy photons to activate the cross-linking agent, in contrast to multiphoton excitation. The activation system 520 may also include a guidance system 524 to ensure the laser system 522 is properly focused and the activating light is delivered to the treated target eye tissue 2 with the proper power/beam intensity. For example, the guidance system 524 may determine the position of the corneal surface, e.g., via captured images, reflected light, etc., and use the corneal surface as a reference to determine the distance from the laser system 522 to the target eye tissue 2. It is understood that any technique may be employed to determine the distance from the laser system 522 to the target eye tissue 2 in order to focus the laser system 522 and to apply the activating light with the proper power/beam intensity.

As described above, the aqueous humor is filtered through the trabecular meshwork (TM) and then drained through Schlemm's canal. Increased aqueous humor outflow resistance in the trabecular meshwork results in elevated IOP. Increased deposition of extracellular matrix (ECM) material within the TM can cause this outflow resistance. Transforming growth factor beta (TGF-β) is a cytokine known to be involved in cell growth inhibition, embryogenesis, differentiation, wound healing and apoptosis in part. In particular, TGF-β can increase ECM deposition in the TM. As such, elevated levels of TGF-β have been associated with increased aqueous outflow resistance in the TM and thus elevated IOP. Accordingly, to address elevated IOP, further embodiments may also down regulate TGF-β to reduce the effects of ECM deposition in the TM.

FIG. 6 illustrates an example system 600 for treating glaucoma. The treatment system 600 includes a cannula body 602 that can be guided to the area of Schlemm's canal to deliver drugs/treatments. The cannula body 602, for example, may be approximately 300 μm in diameter. Aspects of the drugs/treatments delivered by the treatment system 600 promote outflow of aqueous humor through the TM to reduce IOP. According to aspects of the present invention, some of these drugs/treatments may down regulate TGF-β to reduce the effects of ECM deposition.

As shown in FIG. 6, the cannula body 602 may be coupled to respective sources 610a-f of hyaluronic acid (HA), mitomycin C (MMC), prostaglandin analogues (e.g., travoprost, latanoprost), photosensitizer (e.g., riboflavin), oxygen (O₂), and saline (NaCl). Once the cannula body 602 is guided to the area of Schlemm's canal, external pumps can act with the sources 610a-f to flush, soak, and oxygenate the area of Schlemm's canal. The cannula body 602, for example, may be micro-perforated to deliver the drugs/treatments via micro-fluidic mechanisms.

Hyaluronic acid (HA) is one of the major components of the ECM and its deficiency has been associated with outflow resistance through the TM. Mitomycin C controls scarring and can prevent closure of filtration through the TM. Meanwhile, prostaglandin analogues, such as travoprost and latanoprost, can down regulate TGF-β. Travoprost has also been shown to expand the lumens of Schlemm's canal. By applying these drugs, the treatment system 600 improves the outflow of aqueous humor through the TM and into Schlemm's canal.

After the drugs/treatments above have been applied to the area of Schlemm's canal, the photosensitizer, such as riboflavin, produces cross-linking activity to stabilize and maintain the changes that improve the outflow of the aqueous humor. Correspondingly, the treatment system 600 also includes one or more illumination sources to photo-activate the photosensitizer. As shown in FIG. 6, the treatment system 600 includes a near infrared (NIR) illumination source 620a and an ultraviolet A (UVA) illumination source 620b for generating cross-linking activity in the area of Schlemm's canal. An illumination guide, e.g., a diffusing fiber 604, extends through (or otherwise along) the cannula body 602 to deliver the photo-activating light from the illumination sources 620a, b. In addition to stabilizing the effects of the prior application of drugs/treatments, the cross-linking activity may also produce localized shrinkage in the area of Schlemm's canal, and this localized shrinkage may stretch the TM and render the TM more porous to reduce aqueous resistance even further.

In some embodiments, the light diffusing fiber 604 may be approximately 200 μm in diameter. The light diffusing fiber 604 may be similar, for example, to the Corning® Advanced Optics Fibrance™ Light Diffusing Fiber. The treatment system 600 thus cannulates the light diffusing fiber 604 to allow the photo-activating light to illuminate the area of Schlemm's canal with greater than 90% uniformity. In general, light can be emitted longitudinally along the light diffusing fiber 604 and radially from the light diffusing fiber 604 (and through the cannula body 602). In some embodiments, however, the photo-activating light from the light diffusing fiber 604 may be directed toward selected tissue/structures, while the photo-activating light is shielded from other tissue/structures that may be more sensitive. For example, the treatment system 600 may include a physical mask that blocks transmission of the photo-activating light in certain directions, so that when properly oriented, the treatment system 600 does not transmit the photo-activating light to the sensitive structures.

Like the other embodiments described herein, the treatment system 600 may also include a monitoring system that may be employed to monitor the operation of the treatment system 600 and the effects of the treatment. For example, the monitoring system may measure the effects of the cross-linking activity on the strength of the targeted tissue. In some embodiments, this measurement can be made non-invasively and in real time. Additionally, the treatment system 600 may include a controller to control aspects of the operation of the treatment system 600. The controller may be communicatively coupled to the monitoring system to process the images, data, etc., from the monitoring system and to determine any necessary response to such feedback, e.g., in real time.

The embodiments above include controllers for providing various functionalities to process information and determine results based on inputs. Generally, the controllers may be implemented as a combination of hardware and software elements. The hardware aspects may include combinations of operatively coupled hardware components including microprocessors, logical circuitry, communication/networking ports, digital filters, memory, or logical circuitry. The controller may be adapted to perform operations specified by a computer-executable code, which may be stored on a computer readable medium.

The controllers may be a programmable processing device, such as an external conventional computer or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments of the present disclosure, as is appreciated by those skilled in the computer and software arts. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present disclosure can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

Although the embodiments described above may employ riboflavin as a cross-linking agent, it is understood that other substances may be employed as a cross-linking agent. Thus, an embodiment may employ Rose Bengal (4,5,6,7-tetrachloro-2′,4′,5′,7′-tetraiodofluorescein) as a cross-linking agent. Rose Bengal has been approved for application to the eye as a stain to identify damage to conjunctival and corneal cells. However, Rose Bengal can also initiate cross-linking activity within corneal collagen to stabilize the corneal tissue and improve its biomechanical strength. Like riboflavin, photoactivating light may be applied to initiate cross-linking activity. The photoactivating light may include UV light or green light.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

What is claimed is:

1. A system for treating glaucoma, comprising:
a cannula body configured to be positioned in an area of Schlemm's canal;
an illumination guide extending along the cannula body;
at least one drug source coupled to the cannula body, the at least one drug source including a drug that promotes outflow of aqueous humor through the trabecular meshwork and into Schlemm's canal;
a cross-linking agent source coupled to the cannula body; and
an illumination source coupled to the illumination guide, wherein the cannula body delivers the drug from the at least one drug source to the area of Schlemm's canal, and in response to changes in the outflow of aqueous humor, delivers the cross-linking agent to the area of Schlemm's canal, and
the illumination guide delivers photo-activating light from the illumination source to the area of Schlemm's canal after the cross-linking agent has been delivered, the photo-activating light generating cross-linking activity in the area of Schlemm's canal by activating the cross-linking agent, thereby stabilizing changes in the area of Schlemm's canal.

2. The system of claim 1, wherein the at least one drug source includes a drug that down regulates transforming growth factor beta (TGF-β).

3. The system of claim 1, wherein the illumination guide is a light diffusing fiber.

4. The system of claim 1, wherein the cannula body is micro-perforated to deliver the drug from the at least one drug source and the cross-linking agent via micro-fluidic mechanisms.

5. A system for treating glaucoma, comprising:
a catheter configured for insertion into an eye, the catheter having a proximal end and a distal end and including:
a lumen configured to be coupled, at the proximal end, to a cross-linking agent source containing a cross-linking agent formulation;
an optical fiber configured to be coupled, at the proximal end, to a photoactivating light source; and
a guide that is operable from the proximal end to direct the distal end of the catheter to selected eye tissue in a lamina cribrosa, a peripapillary sclera, or a combination of the lamina cribrosa and the peripapillary sclera of the eye as the catheter is inserted into the eye,
wherein the lumen delivers the cross-linking agent formulation from the cross-linking agent source directly to the selected eye tissue at the distal end of the catheter, and the optical fiber delivers photoactivating light from the photoactivating light source directly to the selected eye tissue treated with the cross-linking agent to generate cross-linking activity in the selected eye tissue.

6. The system of claim 5, wherein the guide includes one or more guidewires that extend from the proximal end to the distal end of the catheter, the one or more guidewires being operable from the proximal end to bend the catheter and direct the distal end to the selected eye tissue as the catheter is inserted into the eye.

7. The system of claim 5, further comprising a monitoring system that provides information on a position of the distal end of the eye.

8. The system of claim 7, wherein the monitoring system captures and displays images from the distal end of the eye.

9. The system of claim 7, wherein the monitoring system includes an ultrasound imaging system.

10. The system of claim 7, wherein the monitoring system includes an optical coherence tomography (OCT) system that determines one or more three-dimensional profiles of the selected eye tissue.

11. The system of claim 10, wherein the one or more three-dimensional profiles from the OCT system determines provides information on the biomechanical strength of the selected eye tissue.

12. The system of claim 10, wherein the one or more three-dimensional profiles from the OCT system are determined in response to a perturbation of the selected eye tissue.

13. The system of claim 5, wherein the cross-linking agent source is a syringe including a plunger and reservoir, the reservoir containing the cross-linking agent formulation, the plunger being operable to push a dose of the cross-linking agent through the lumen to the distal end of the catheter.

14. The system of claim 5, further comprising a controller that controls the photoactivating light source according to at least one of the following parameters:
wavelength, bandwidth, intensity, power, or duration.

15. A system for treating glaucoma, comprising:
a cross-linking agent system including a cross-linking agent source containing a cross-linking agent formulation and a cross-linking agent delivery device, the cross-linking agent delivery device being coupled to the cross-linking agent source, the cross-linking agent delivery device being configured for insertion into the eye and to deliver the cross-linking agent formulation from the cross-linking agent source directly to selected eye tissue in a lamina cribrosa, a peripapillary sclera, or a combination of the lamina cribrosa and the peripapillary sclera of an eye; and
an activation system including a photoactivating light source and a photoactivating light delivery device configured to deliver photoactivating light to the selected eye tissue treated with the cross-linking agent formulation to generate cross-linking activity in the selected eye tissue.

16. The system of claim 15, wherein the activation system is configured to deliver the photoactivating light from outside the eye and to a depth of the selected eye tissue below a surface of the eye.

17. The system of claim 15, wherein the activation system includes a laser system.

18. The system of claim 17, wherein the activation system includes a mirror array to determine a pattern for the photoactivating light from the laser system and an objective lens to determine a focal depth for the pattern of photoactivating light.

19. The system of claim 17, wherein the activation system provides multiphoton excitation with the laser system.

20. The system of claim 15, further comprising a controller that controls the photoactivating light source according to at least one of the following parameters: wavelength, bandwidth, intensity, power, or duration.
